(19) United States
(12) Patent Application Publication
(10) Pub. No.: US 2016/0250331 A1
(43) Pub. Date: Sep. 1, 2016

(54) Biodegradable Magnetic Nanoparticles and Related Methods

(71) Applicant: REGENTS OF THE UNIVERSITY OF MINNESOTA, Minneapolis, MN (US)

(72) Inventors: Jian-Ping Wang, Minneapolis, MN (US); Ying Jing, Minneapolis, MN (US); Shihai He, Minneapolis, MN (US)

(21) Appl. No.: 15/059,048

(22) Filed: Mar. 2, 2016

Related U.S. Application Data
(63) Continuation of application No. 13/708,658, filed on Dec. 7, 2012, now abandoned.
(60) Provisional application No. 61/567,984, filed on Dec. 7, 2011.

(51) Int. Cl. A61K 41/00; A61K 49/18

(52) U.S. Cl. CPC A61K 41/0052; A61K 49/1818

(57) ABSTRACT

The design of biodegradable magnetic nanoparticles for use in in-vivo biomedical applications. The particles can include Fe in combination with one or more of Mg, Zn, Si, C, N, and P atoms or other particles. The nanoparticles can be degraded in-vivo after usage. The nanoparticles can cease heating upon reaching a predetermined temperature or other value.

### REFERENCE TO RELATED APPLICATIONS

This application is a continuation of U.S. application Ser. No. 13/708,658 filed Dec. 7, 2012, which claims priority under 35 U.S.C. 119(e) of U.S. Provisional Application Ser. No. 61/567,984 filed Dec. 7, 2011, which are incorporated herein by reference.

### GOVERNMENT SUPPORT

This invention was made with government support under BME 0730825 and DMR-0819885, both awarded by the National Science Foundation. The government has certain rights in the invention.

### BACKGROUND

Magnetic nanoparticles are being widely investigated for bio-medical applications. The possibility to manipulate and control magnetic property of magnetic nanoparticles leads to diverse applications in diagnosis, disease treatment and even disease detection. As more and more uses of nanoparticles for in-vivo applications emerge, concerns on their toxicity are raised. For example, quantum dots with proper organic coating are found highly stable inside the body and show fluorescent property for almost two years. To address toxicity concerns, new magnetic nanoparticles which can safely be cleared out of human body within an acceptable time period are desirable.

Biodegradable implants/stents have long been explored and used. They have specific applications on tissue repair, bone support, surgery and so on. Biodegradable nanoparticles are to be used for imaging, cell tracking, drug delivery, cancer therapy et al. A few attempts on making biodegradable nanoparticles are reported. For instance, luminescent porous silica particles in micrometer size are found degradable with low cytotoxicity. Composite particles made of 4-5 nm Au nanoparticles are also claimed biodegradable since they decompose into small clusters that then get cleared out from the body. Although iron oxide magnetic nanoparticles are sometimes considered degradable, belief in this is not fully established. In then get cleared out from the body. Although iron oxide magnetic nanoparticles are sometimes considered degradable, belief in this is not fully established. In addition, residence time of iron oxide nanoparticles inside the body is long. In addition, iron oxides are not superior in terms of their magnetization, namely magnetic signal per unit volume. Degradation rate is not very adjustable due to the fixed chemical composition previously used.

In addition, there has been burgeoning interest in magnetic hyperthermia because of its potential on cancer treatment with less side-effect. The technique takes advantage of low heat endurance of malignant cells compared to normal cells. Under high frequency AC magnetic field excitation, heat released from magnetic nanoparticles would lead to degrading of malignant cells. Although nano-scale magnetic nanoparticles have been applied widely to produce localized heat in near proximity of targeted tissue, intercellular heating by multiple nanoparticles is more feasible. With a large number of heat source spreading around the targeted area, working efficiency is expected to be high. In this circumstance, precise control of temperature in the safe working range is a challenge. How localized can the heat profile be, and how accurate can the device sense in-vivo temperature and control on-off are bottleneck issues for traditional magnetic hyperthermia.

Proposal of candidate materials for self-regulated magnetic hyperthermia is emerging. People aim at using the ferromagnetic transition temperature to achieve self-regulation. Magnetic materials with Curie temperature close to safe working range 42° C.-49° C. have been investigated, including Ni doped Cu, La₁₋ₓSrₓMnO₃, Fe—Ni based alloy and Zn ferrite. Although the materials have suitable Curie temperature, most of them have low saturation magnetization, which affects heating efficiency greatly. There are also concerns of the biocompatibility of these materials.

Previously magnetic nanoparticles are engineered with different shape and size with different composition by using different synthesis approaches.

### SUMMARY

In one or more embodiments, nanoparticles include magnetic biodegradable biocompatible nanoparticles. For example, in one or more embodiments, the nanoparticles include one or more of Fe—Zn, Fe—Mg, Fe—Ca, Fe—Si, Fe—C, Fe—N, Fe—P or their mixed (Fe—Si—Zn—Mg—N— . . . ) amorphous, partial-crystalline alloy and alloy nanoparticles. The magnetic nanoparticles could have higher or comparable magnetic moment than traditional iron oxides. In one or more embodiments biodegradability is tuned through alloy composition. In one or more embodiments, the resultant biodegradation products are Fe, Zn, Mg, Si, C, P, N and Ca ions based, which all make up basic trace elements in human body.

The embodiments further include choosing the biocompatible atoms and 1) engineering them into magnetic nanoparticles by adjusting the chemical binding between the atoms to enable its biodegradability and 2) tuning the exchanging coupling constant, a quantum mechanical parameter, between the atoms to enable its temperature self-regulation response for in-vivo applications.

### BRIEF DESCRIPTION OF THE FIGURES

FIG. 1a illustrates a schematic diagram of Fe—Zn alloy nanoparticles in accordance with one or more embodiments.

FIG. 1b illustrates a schematic diagram of Fe—Zn nanoparticles with Fe clusters embedded in Zn matrix in accordance with one or more embodiments.

FIG. 1c illustrates a schematic diagram of mesoporous Fe—Zn nanoparticles with aggregated core-shell small clusters in accordance with one or more embodiments.

FIG. 1d illustrates a schematic diagram of Fe—Zn core shell nanoparticles in accordance with one or more embodiments.

FIG. 1e illustrates a schematic diagram of Fe—Zn core multi-shell nanoparticles in accordance with one or more embodiments.

FIG. 1f illustrates a schematic diagram of a nanotube/nanobelt embedded with Fe nanoparticles in accordance with one or more embodiments.

FIG. 2 illustrates a schematic diagram of in-vitro Biodegradation Test Flow in accordance with one or more embodiments.

FIG. 3 illustrates a chart showing a change of magnetic moment of Fe—Zn nanoparticles as a function of time in accordance with one or more embodiments.

FIG. 4 illustrates a block diagram of an apparatus for a FeZn nanoparticle fabrication system constructed in one or more embodiments.

FIG. 5a illustrates alloy structure of FeZn fabricated by tube target nanoparticle deposition system in accordance with one or more embodiments.

FIG. 5b illustrates mesoporous structure of FeZn fabricated by tube target nanoparticle deposition system in accordance with one or more embodiments.

FIG. 5c illustrates cluster matrix structure of FeZn fabricated by tube target nanoparticle deposition system in accordance with one or more embodiments.

FIG. 6a illustrates a block diagram of an apparatus for a FeMg fabrication system constructed in one or more embodiments.

FIG. 6b illustrates a TEM image of a FeMg nanoparticle in accordance with one or more embodiments.

FIG. 7a illustrates a bright field TEM image of Fe₅Si₃ nanoparticles in accordance with one or more embodiments.

FIG. 7b illustrates a high resolution TEM image of a single Fe₅Si₃ nanoparticle in accordance with one or more embodiments.

FIG. 8a illustrates a HADDF image of Fe₅Si₃ nanoparticles in accordance with one or more embodiments.

FIG. 8b illustrates a core of one Fe₅Si₃ nanoparticle in accordance with one or more embodiments.

FIG. 8c illustrates a shell of one Fe₅Si₃ nanoparticle in accordance with one or more embodiments.

FIG. 8d illustrates an XRD pattern of an ensemble of as synthesized Fe₅Si₃ nanoparticles; in accordance with one or more embodiments.

FIG. 9a illustrates a hysteresis loop for Fe₅Si₃ nanoparticles at room temperature in accordance with one or more embodiments.

FIG. 9b illustrates experimental and fitting curves based on initial magnetization measurement in accordance with one or more embodiments.

FIG. 9c illustrates magnetization of Fe₅Si₃ nanoparticles as a function of temperature in an external field of 1 T in accordance with one or more embodiments.

FIG. 10 illustrates ICP results of Fe and Si concentration in the supernatant of a solution of Fe5Si3 nanoparticles for different time period in accordance with one or more embodiments, where the solution was treated by simulated biological condition: PH=5 buffer and constant 37 Celsius degree.

FIG. 11 illustrates TEM images of Fe—Si NPs at 42 at % Si in accordance with one or more embodiments.

FIG. 12a illustrates T_{c} of Fe—Si nanoparticles as a function of atomic percentage of Si in accordance with one or more embodiments.

FIG. 12b illustrates temperature dependence of magnetization from 5K to 300K in accordance with one or more embodiments.

FIG. 12c illustrates fitting of curves in FIG. 12b based on Bloch's law in accordance with one or more embodiments.

FIG. 12d illustrates spin stiffness extrapolated from experimental fitting for Fe—Si particles of different composition in accordance with one or more embodiments.

FIG. 13a illustrates a characterization of Fe—Si NPs including a bright field TEM image of Fe—Si NPs in accordance with one or more embodiments.

FIG. 13b illustrates a characterization of Fe—Si NPs including a SAD of Fe—Si NPs in accordance with one or more embodiments.

FIG. 13c illustrates a characterization of Fe—Si NPs including a hysteresis loop of Fe—Si NPs in accordance with one or more embodiments.

FIG. 13d illustrates a characterization of Fe—Si NPs including a temperature dependence of magnetization in accordance with one or more embodiments.

FIG. 14a illustrates the cytotoxicity of Fe—Si NPs including a, Viability of mouse embryonic fibroblasts (NIH 3T3) after treatment with Fe—Si NPs at different concentrations in accordance with one or more embodiments.

FIG. 14b illustrates the cytotoxicity of Fe—Si NPs including cytotoxicity result of Fe—Si NPs on HUVECs in accordance with one or more embodiments.

FIG. 15 illustrates dye release under magnetic field heating by POEA/Fe—Si NPs composite showing temperature rise versus time for POEA block copolymer loaded with Fe—Si NPs and dyes, and POEA block copolymer loaded with dyes only in accordance with one or more embodiments.

FIG. 16 illustrates hemolysis results of Fe—Si NPs in accordance with one or more embodiments.

FIG. 17 illustrates temperature rise v. time of a water solution of Fe Si NPs in accordance with one or more embodiments.

### DETAILED DESCRIPTION

This application discusses the design of magnetic nanoparticles, including magnetic biodegradable nanoparticles that can play an important role for various in-vivo biomedical applications, including, but not limited to MRI contrast agents, drug carriers, or magnetic hyperthermia. In one or more embodiments, a composition includes a biocompatible and biodegradable nanoparticle including amorphous, partial-crystalline and crystalline alloy or structure, and the nanoparticle includes an alloy of Fe and at least one of Mg, Zn, or Si. In one or more embodiments, the amorphous, partial-crystalline or crystalline alloy has more than 30 at % Fe. In one or more embodiments, a composition comprises a biocompatible and biodegradable nanoparticle including at least one of amorphous alloy, partial-crystalline alloy or crystalline alloy structure, and the nanoparticle includes an alloy of Fe and at least one of Mg, Zn, or Si. In one or more embodiments, chemical binding between these atoms in nanoparticles is adjusted by the synthesis process to enable the different crystallinity of the particles, which will control their biodegradability.

In one or more embodiments, nanoparticle includes at least one of an amorphous or partial-crystalline or crystalline alloy of Fe with at least one of Mg, Zn or Si mixed with elements of at least one of N, P, S, C, Ca, Ag, or Mn. In one or more embodiments, the nanoparticle is a heterostructure having a structure and a matrix, the structure includes at least one of FeSi, FeZn, FeMg, FeN, FeC or FeP, and the matrix includes one or more of Fe, Si, P, N, C, P, Ag. Mn. In one or more embodiments, the nanoparticle includes a heterostructure having at least one of Fe clusters, Fe alloy clusters, or Fe—Mg, Zn, Si, N, P, C core-shell clusters embedded in Mg, Zn, Si or a corresponding matrix. In at least one embodiment, the nanoparticle includes at least one of Fe—Mg, Zn, Si, N, P, or C core-shell crystals coalesce and form at least one of a mesoporous composite, or at least one of a nanobelt or nanotube embedded with Fe particles or Fe alloy particles. In at least one embodiment, the nanoparticle includes a heterostructure having Fe core or at least one of a Fe—Mg, Zn, Si, N, P, C amorphous or alloy core, multiple shell layers of different material composition on the nanoparticle, the shell layer different than the Fe core or Fe—Mg, Zn, Si, N, P, C amorphous or alloy core. Optionally, the nanoparticle has one or more of a nanotube, nanosphere, nanorod, nanodisk, hollow rod, or cylinder shape.

In an embodiment, the nanoparticles include Fe—Zn and/or Fe—Mg metallic nanoparticles that are biodegradable and biocompatible nanoparticles. The nanoparticles can also be used for drug delivery, drug delivery with self-regulated release, cancer treatment, and/or thermal ablation, MRI contrast agents. The nanoparticles are biodegradable and biocompatible high-magnetic-moment nanoparticles for large signal and little long-term side-effects. The time period for complete degradation can be a few weeks to several months, and the products are non-toxic and do not disturb cell level functions. The degradation products are excreted out, for example. In one or more embodiments, the nanoparticle has a high magnetic moment and a sharp transition for magnetization v. temperature. In at least one embodiment, the nanoparticle is a self-regulating magnetic hyperthermia particle.

In one or more embodiments, the nanoparticles include magnetic biodegradable biocompatible nanoparticles. For example, in one or more embodiments, a composition includes nanoparticles that include one or more of Fe—Zn, Fe—Mg, Fe—Ca, Fe—Si, Fe—Ca, Fe—C, Fe—N, Fe—P or their mixed (Fe—Si—Zn—Mg—N— . . . ) metallic nanoparticles. The magnetic nanoparticles could have higher or comparable magnetic moment than traditional iron oxides. In one or more embodiments biodegradability is tuned through alloy composition. In one or more embodiments, the resultant biodegradation products are Fe, Zn, Mg, Si, C, P, N and Ca ions based, which all make up basic trace elements in the human body.

Magnetic nanoparticles can play an important role for various in-vivo biomedical applications such as MRI contrast agents, drug carriers, magnetic hyperthermia etc. Investigation into semiconductor, noble metal nanoparticles revealed high possibility of sequestering inside lung and spleen, which is unfavorable for any type of in-vivo applications. Size dependent clearance was found while medium size nanoparticles are more unlikely to be cleared out. Present status puts biodegradable nanomaterials to an important aspect. Degradation of nanoparticles will lead to clearance of them out of human body instead of sequestering inside. The clearance scheme can be either reduction of size into the favorable regime of renal type clearance, or gradual decomposing into ions without any integrated entities existing. The time period for degradation can be a few weeks to several months. The time period is important for keeping biological life on normal level. Too fast degradation rate might cause inflammation, higher cytotoxicity, but too slow degradation rate still faces the danger of sequestering nanoparticles. Control of degradation rate should be done by engineering material composition, material structure and surface chemistry. For example, in a binary alloy the degradation rate can be raised by increasing the composition ratio of the component with higher degradation rate. Besides, the materials with amorphous structure tend to have higher degradation rate than the materials with crystallized structure. When degradation occurs, it should result in non-toxic results and undisturbed cell level functions. The overall life activities should be unaffected and remain normal. Degradation of nanoparticles is driven by chemical reaction when the nanoparticles are exposed to biological fluid environment. The reaction process must not produce free radicals which are cytotoxic to human body. The reaction process also must not catalyze or suppress other reactions. The product function should include participation in biological activities, and the products are excreted out by the patient.

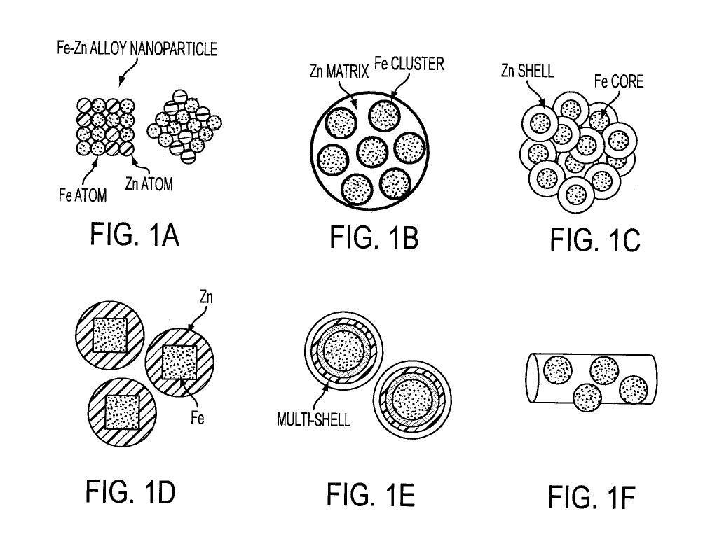

One type of structure that could be used as in-vivo biodegradable nanomaterial is solid solution of Fe and other elements which have low melting temperature, low surface energy and good solubility in water. (FIG. 1(a)) In this type, a relatively fast degradation rate is the target. The substitution of atoms can lead to point defects. Bonding among atoms is prone to be broken in acid fluidic environment through charge transfer.

Another type of biodegradable structure can be metal/nitride or phosphorous matrix with Fe or Fe amorphous or Fe alloy or Fe compound clusters embedded inside. Size of one nanoparticle might be large. Within one nanoparticle, small Fe clusters reside in the matrix (See FIG. 1(b) for example). The outer matrix with fast degradation rate gradually decomposes and releases ions. Individual Fe clusters of smaller size are easy to be excreted out by patients' body. Formation of this type of composite structure is based on high matrix material concentration (40 at %-80 at %) and diffusion driven process. The duration for degradation and clearance to complete is longer for this type due to the existence of oxide and elemental metal.

Mesoporous type of nanoparticles consisting of small crystallites is an alternative of the cluster-matrix type. The mesoporous type of nanoparticles has small crystallites as basic units and doesn't have a solid matrix (See FIG. 1(c) for example). Formation of this type of composite is possible in gas phase synthesis method. Small core-shell crystallites (eg. Fe—Zn, Fe—Mg) or alloy crystallites carry out second growth through oriented attachment or agglomeration. Stopping growth in an intermediate stage, mesoporous composite is obtained before re-crystallization takes place. This type of biodegradable material possesses large surface area for loading biological subjects. Biodegradation proceeds by decomposing into the small building units.

Other heterostructure might also be developed to make magnetic nanoparticles biodegradable. For example, core-shell type of nanoparticles with highly magnetic core protected by a multiple shell layers of different material composition than the Fe core or Fe alloy core, where for example, a biodegradable shell is a less complex structure. (FIG. 1 (d)) Multi-shell or multi-layered nanoparticles with different shells/layers where each layer has its own degradation rate are an additional type. (FIG. 1(e)) This provides more control of degradation process according to the need. A dumb-bell/branch like heterostructure with each part of different degradation rate and magnetic/optical property can offer a broad range of multifunctional capability. Hollow nanoparticles with biodegradable magnetic material making up the shell can be very useful for biomedicine. Different shapes for the nanoparticle and/or the shell can also be developed for various applications, for example, nanotube, nanoshpere, nanobelt, nanorod, nanodisk, hollow rod, or cylinder shape, and so on. Different size of nanoparticles can also influence the clearance time of them inside human body.

The magnetic biodegradable nanoparticles include, but are not limited to, Fe—Zn and Fe—Mg metallic nanoparticles. The above mentioned alloys, composite structures are applicable to these material systems. The nanoparticles could have higher or comparable magnetic moment than traditional iron oxides. The biodegradability can be tuned, in an option, via composition. The biodegradability can also be tuned via composite structure. The biodegradability can also be tuned via crystal structure by engineering the chemical binding between the atoms within nanoparticles. Either amorphous or partial crystalline or crystalline structure is possible for biodegradable magnetic nanoparticles. When the structure approaches amorphous, degradation rate could be even enhanced due to weak bonds among atoms. The biodegradation productions are Fe, Zn, and Mg ions based, which are basic trace elements in the human body.

In a system using Fe—Zn particles, the particles can be made using sputtering-based gas phase condensation, mechanical alloying, electro-deposition, or chemical methods. When used in biomedical applications, the Fe—Zn particles include a structure of Fe or FeZn clusters that are embedded in a Zn matrix, or an aggregated structure with Fe—Zn Core-Shell small clusters A high magnetic moment comes from the α-Fe, and biodegradability comes from the Zn matrix. Zn doping in Fe could reduce the exchange coupling between Fe atoms, a quantum parameter, to determine its Curie temperature. Thus Zn doped FeZn clusters could possess the adjustable Curie temperature that could be engineered to be very close to room temperature. This feature allows us form self-regulated biodegradable magnetic nanoparticles.

In a system using Fe—Mg particles, the particles can be made using sputtering-based gas phase condensation or mechanical alloying techniques. For example, co-sputtering Fe and Mg includes up to 7% Mg films of solid solution. When the Mg content goes beyond 30%, the film is completely amorphous. Previously, thin films of Fe and Mg can form solid solution by sputtering but only with low solubility. For the technique of mechanical alloying, crystalline Fe—Mg nanoparticles can be successfully synthesized with higher Mg solubility. Saturation magnetization change follows linear dilution caused by Mg. Mechanical alloying can be carried out by high-energy ball milling with surfactant. There was also chemistry method to make Fe—MgO core-shell type of nanoparticles. So the two materials can have different combination to make a structure that fits into application requirement. The degradation rate can be easily controlled by changing the composition of the Fe—Mg nanoparticle which is decided by the sputtering target being used. In gas phase synthesis process using planer targets, formation of nanoparticles is governed mostly by kinetic process. There is a chance to make higher solubility crystalline Fe—Mg by obtaining nanoparticles out of snapshots. However, it is not limited to only alloy type nanoparticles in order to have biodegradable nanomaterial for in-vivo use.

The two elements have very limited solubility in each other. BCC phase or HCP phase can be formed at low Mg (<20%) concentration or low Fe concentration (<20%), respectively. Mg or Fe atoms will occupy substitutional positions to form the alloy. Composite with both phase structures is expected for the intermediate composition. In an option, for example for biomedical application, low Mg concentration with less than about 20% Mg is desirable to form FeMg alloy with Mg substitution. Magnetization of the material is high (>178 emu/g, comparing to this magnetite has a magnetization of 84 emu/g) and degradation behavior can be adjusted by changing the composition ratio of Mg. Typically higher Mg composition ratio leads to higher degradation rate as the Mg site is more active to the acid fluidic environment in cells. Mg doping in Fe could reduce the exchange coupling between Fe atoms, a quantum parameter, to determine its Curie temperature. Thus Mg doped FeMg clusters could possess the adjustable Curie temperature that could be engineered to be very close to room temperature. This feature allows us form self-regulated biodegradable magnetic nanoparticles.

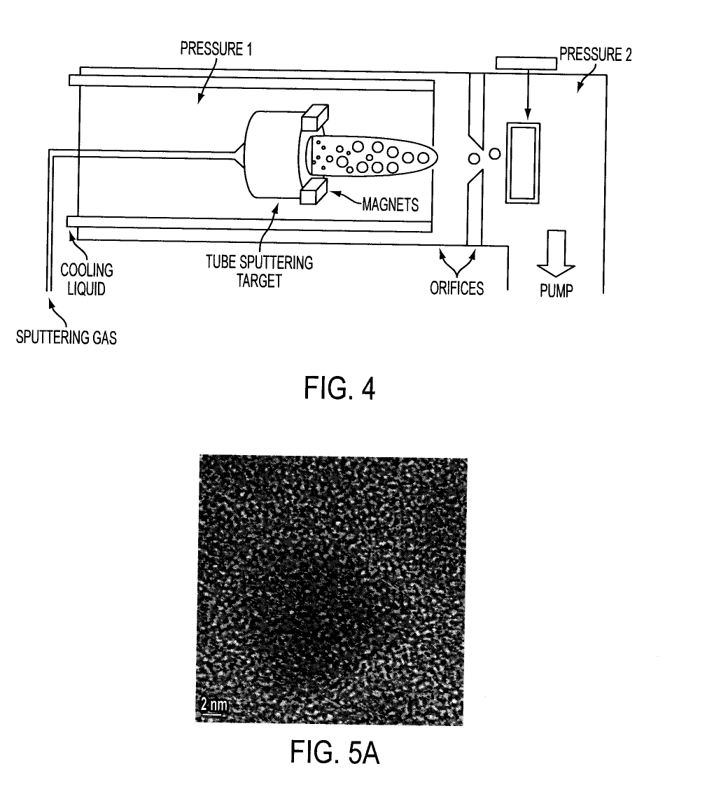

FIG. 4 illustrates an example of a FeZn NPs fabrication system. In an example, Fe₅₀Zn₅₀ tube target (40 mm tall, 20 mm outside diameter and 5 mm inside diameter, the size could change accordingly), is used as the cathode in the sputtering-based nanoparticle fabrication system. During fabrication process, high negative voltage is applied to the tube target and Ar sputtering gas is injected through the tube target hole. The high negative voltage ionize the Ar gas to generate Ar+ ions which will be accelerated to hit the inside wall of the target to knock out the Zn and Fe atoms. Then the Zn and Fe atoms having been knocked out will be carried out of the target to form high density atoms. At high pressure environment, the atom gas condenses to form FeZn nanoparticles. Sputtering pressure is 500 mTorr to 2 Torr, sputtering power is 100 W to 400 W. Other Fe—Zn, Fe—Mg, Fe—Ca, Fe—Si, Fe—C, Fe—N, Fe—P nanoparticles can also be synthesized by this fabrication system, using Fe—Zn, Fe—Mg, Fe—Ca, Fe—Si, Fe—C, Fe—N, Fe—P tube targets. The composition of the target materials will be decided by the desired composition of the synthesized binary nanoparticles.

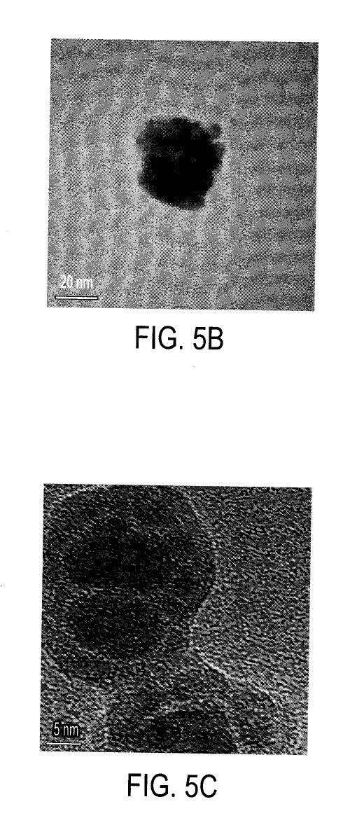

FIGS. 5a-5C illustrates particles formed by the tube target apparatus of FIG. 4 according to one or more embodiments. FIG. 5a illustrates alloy structure of FeZn fabricated by tube target nanoparticle deposition system. FIG. 5b illustrates mesopourous structure of FeZn fabricated by tube target nanoparticle deposition system. FIG. 5c illustrates cluster matrix structure of FeZn fabricated by tube target nanoparticle deposition system.

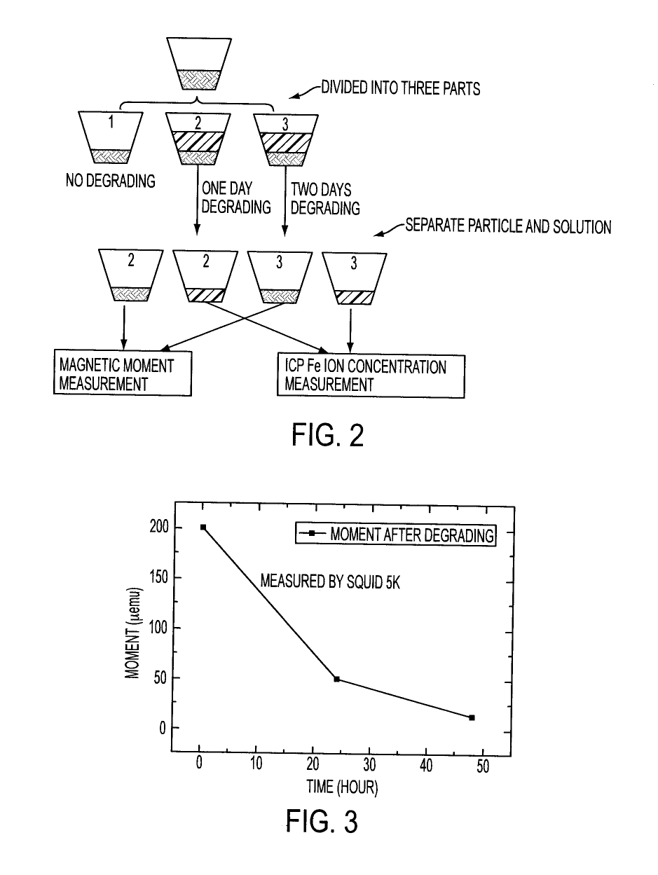

An in-vitro experiment was conducted to test the degradability of Fe—Zn nanoparticles. The procedure was illustrated in FIG. 2. FeZn nanoparticles were deposited using the above mentioned technique onto PEG coated glass slides and transferred into water. They were then treated by PH=7.4 PBS buffer and 37 Celsius degree water bath conditions, a simulated environment of human body fluid. In different time intervals, un-dissolved nanoparticles were collected by centrifugation and magnetic moment of them was measured by SQUID at 5K. FIG. 3 shows the change of the magnetic moment of Fe—Zn nanoparticles under in-vitro condition as a function of time. Decrease of magnetic moment indicates degradation of Fe—Zn nanoparticles under simulated human body fluid environment. Fe and Zn ions continuously dissolved into water solvent and no longer contributed to the magnetic signal. Complete degradation took place within 3 days for this particular case.

Many properties of the particles can be modified by alloy selection. For instance, the magnetic moment can be changed by changing the composition ratio of the iron for alloy type structure. High magnetic moment, equivalent to pure elemental Fe, can be maintained for composite structures. The degradation rate can be adjusted by changing the ratio of Mg or Zn, as well as the heterostructure. By adding a third element, multi-functional nanoparticles could be synthesized. For example, if a semiconductor (such as silicon) small crystal is embedded in the nanoparticle, or if we incorporate porous silica shell layer, they will have luminescence property besides magnetism and biodegradability; it will facilitate the tracking of the nanoparticle. By adding another third element, crystalline structure and crystallinity of the nanoparticles could be controlled. For example, in doping an abundant element in magnetic nanoparticles like N or C or Si in the nanoparticles, the biodegradation rate could be tuned. Nanobelt or nanotube encapsulating small magnetic nanoparticles is another type of biodegradable heterostructure when material such as carbon is incorporated. (FIG. 1(f)) Abundant elements such as P, S and trace element Mn are also present in human body, which are possible doping elements to modify the magnetic property and degradation rate. The nanoparticles are biodegradable and biocompatible for in vivo biomedical applications. The particles can be synthesized by physical gas condensation system with a tube cathode. In one or more embodiments, the FeZn nanoparticles have a Curie temperature above room temperature.

Surface functionalization of biodegradable nanoparticles is needed to make them water soluble and prevent aggregation inside human body. Polyethylene glycol (PEG) is FDA approved, biocompatible polymer for in-vivo use. It has been demonstrated that the polymer can enhance circulation duration time of nanoparticles inside human body, which assists specific tissue or cell targeting. To form covalent bonding between nanoparticles and PEG, 3-Triethoxysilylpropylamine (APTES) modification on the surface of nanoparticles can be performed ahead. Magnetic nanoparticles are deposited using physical gas condensation technique onto PEG (molecular weight 2000) coated glass slides. In this way, nanoparticles can be transferred into aqueous media by washing them off from the glass slides. After washing out extra polymers, APTES modification will introduce amino group onto the surface of nanoparticles. mPEG-NHS or mPEG-NH2 (molecular weight 5000-10000) can be bonded covalently in the presence of DMSO reagent. Further functionalization for targeting, delivery or anti-cancer purpose can be incorporated by employing multi-arm PEG. Polymers instead of PEG, such as glucose, biodegradable thermal sensitive POEG, can also be used for surface functionalization. Besides APTES modification, incorporation of —CHO group onto the surface can be realized through EDC/sulfNH2. Covalent bonds are formed in the presence of —CHO group.

The following are options for the nanoparticle. In an option, Si, N, C, P, S, Ag, Mg, Zn, Mn can be used for doping of Fe for biocompatible and biodegradable magnetic nanoparticles. In one or more embodiments, incorporation of Si crystal or porous silica layer or ZnO layer incorporation to make optically active biodegradable magnetic nanoparticles. Other options regarding biodegradability include the nanoparticles are biodegradable with a tunable degradation rate because of composition, heterostructure characteristic, degree of crystallinity, shape, doping, composition and heterostructure. Further options for the nanoparticle are that they have a high magnetic moment, can be multifunctional with magnetic, optical property combined.

The nanoparticles can be made in several methods. For instance, they can be made by a physical gas condensation method using a circular planar alloy target/composite target, a physical gas condensation method using a tube alloy target/composite target, or can be synthesized under Ar2 sputtering gas or Ar2/N2 mixture sputtering gas.

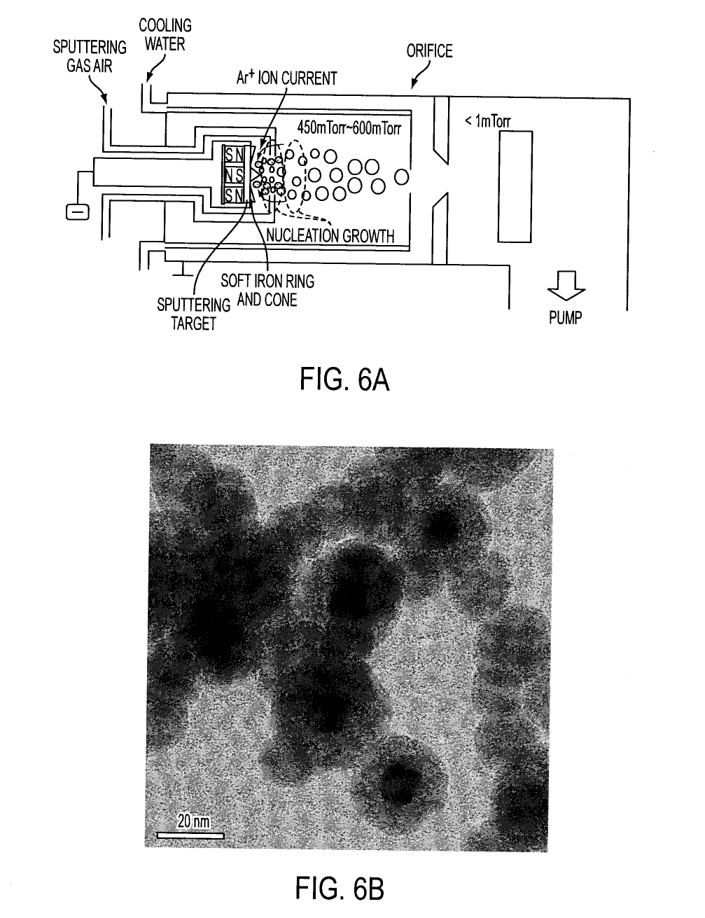

FIG. 6a illustrates an apparatus for making FeMg nanoparticles with planer magnetron sputtering based deposition system. Alloy FeMg targets with disk shape were used for the fabrication, at a sputtering pressure of 450 m Torr and a sputtering current of 0.3 A. During fabrication process, high negative voltage is applied to the target and Ar sputtering gas is injected near the target surface. The high negative voltage being applied on the target ionize the Ar gas to generate Ar+ ions which will be accelerated to hit the planar disk target to knock out the Mg and Fe atoms. Then the Mg and Fe atoms having been knocked out form high density atoms density. At high pressure environment, the atom gas condenses to form FeMg nanoparticles. Sputtering pressure is 200 mTorr to 1 Torr, sputtering power is 50 W to 500 W. FIG. 6b illustrates a TEM image of a FeMg nanoparticle.

Various applications of the nanoparticles include magnetically heating by magnetic, biocompatible and biodegradable nanoparticles described herein, and maintaining a predetermined temperature based on the magnetic property of the nanoparticles. Further options include, but are not limited to, providing high contrast and high signal to noise ratio as MRI imaging agents, acting as non-viral transfection agents carrying and directing gene to targeted position by external magnetic field gradient, carrying and directing drug to targeting position by external magnetic field gradient, remotely controlled releasing drug loaded in nanoparticles through application of AC magnetic field, bonding to or uptaken by stem cells for tracking targeting cell membranes and controlling ion channel or response of cells through magnetic heating or magnetic motion. Additional applications include magnetic nanoparticle imaging of vascular or intestine region based on the nonlinearity of magnetization curve by point-of-care medical devices, drug eluting stents with drug loaded in nanoparticle carriers and achieving controllable release performance through magnetic heating, detecting various pattern of magnetic signal for cell line differentiation, distinct cellular state examination.

In one or more embodiments, the nanoparticles include “smart” nanoparticles able to sense the temperature and stop heating automatically upon reaching a predetermined value. In an embodiment, Si doped FeSi particles, especially Fe₅Si₃ can be used for self-regulated magnetic hyperthermia application based on its advantageous saturation magnetization and biocompatibility. The nanoparticles can also be used for drug delivery, drug delivery with self-regulated release, cancer treatment, and/or thermal ablation. A physical gas condensation method was employed to synthesize Fe₅Si₃ nanoparticles with narrow size distribution successfully. Phase and composition of the nanoparticles were experimentally confirmed. When the decline of anisotropy constant is significant, heating behavior will cease as the heating efficiency has close correlation with the anisotropy constant at a particular AC field. These features might offer a solution to highly temperature sensitive self-regulated magnetic hyperthermia.

As mentioned above, Si doped Fe particle, especially Fe₅Si₃ particle, is proposed as a new candidate for biocompatible self-regulated magnetic hyperthermia. Not only does this ferromagnetic material have suitable Curie temperature at for example 385K, but also it has relatively high saturation magnetization 358 emu/cm³. Other suitable temperatures include about 378K-380K, and a high magnetization of at least 350 emu/cm³. Additionally, it is composed of only benign elements Fe and Si, good for biological use. Temperature dependent anisotropy constant can be employed, which could provide more sensitive heat control.

For an application that may need higher or lower regulation temperature, the size and shape of FeSi particles can be engineered and/or form the core-shell or heterostructure with Si doped Fe (e.g. Fe₅Si₃) as the core or embedded cluster in Fe or Fe₃Si matrix. The Curie temperature can be adjusted by engineering the ratio of the core-shell and other materials in the heteterostructured particle. Si doped Fe particle is also biodegradable at certain condition, especially at a heated condition.

In one or more embodiments, a gas phase method is used to fabricate Fe₅Si₃ nanoparticles. A piece of composite Fe—Si target was located in the high vacuum chamber (1×10⁻⁷ Torr) as sputtering source. Ar₂ gas served as both sputtering gas and carrier gas. Magnetic field on the surface of the target was adjusted by placing a soft iron cone and a ring on the target. In this way, distribution of plasma in space is modified. When cross over the plasma region, nano-clusters “freeze” at the equilibrium phase or non-equilibrium phase corresponding to the temperature and period of time experienced. With the right concentration of Fe and Si, and the suitable thermal environment provided by purposely modified plasma, Fe₅Si₃ nanoparticles were obtained.

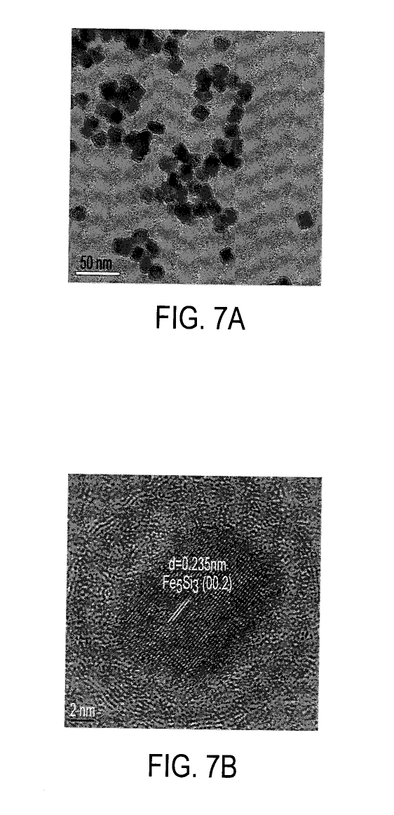

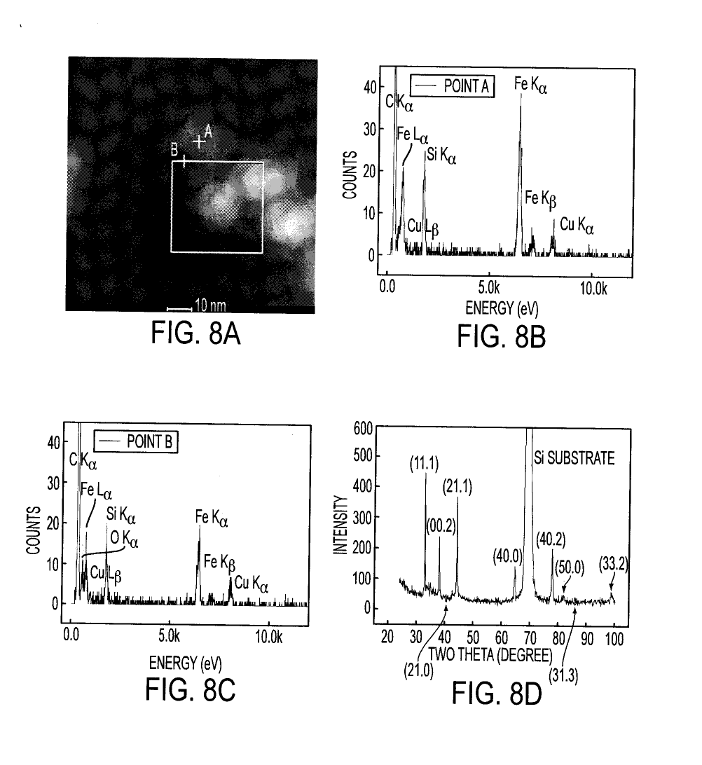

A transmission electron microscope (TEM) was used to characterize the morphology of Fe₅Si₃ nanoparticles. FIG. 7(a) shows uniform nanoparticles with mean size of 18.2 nm, 12.3% standard deviation. High resolution TEM image of a single nanoparticle (FIG. 7(b)) gives clear lattice fringes. Lattice spacing of the planes was measured to be 0.235 nm, which agrees well to the spacing of (00.2) plane of Fe₅Si₃ phase. Presence of Fe and Si elements was investigated by a scanning transmission microscope (STEM) equipped with an energy dispersive X-ray spectrometer (EDS). FIG. 8(a) depicts the high angle annular dark field (HADDF) image of the nanoparticles by STEM. A core-shell structure is indicated by the brighter center and darker edge of nanoparticles. Quantitative composition ratio was measured through EDS spot scan, in which electron beam was spotted on the core and shell (center of cross A and B in FIG. 8(a)) respectively and EDS spectrum was collected (FIG. 8(b) 8(c)). C and Cu peaks in the spectrum come from amorphous carbon coated copper grid substrate. Atomic composition ratio was calculated based on the integrated intensity of K line of Fe and Si after spectrum fitting. The result shows that the core has Fe:Si=1.60, close to 1.67 for Fe₅Si₃, and the shell has Fe:Si=1.06. Thus a structure of Fe₅Si₃ core and oxidized FeSiO shell is confirmed. This explains the brightness difference between center and edge of nanoparticles because contrast in HADDF images reflects atomic number (Z) difference caused change of scattering cross-section. To further confirm the phase of the sample, powder X-ray diffraction (XRD) pattern of Fe₅Si₃ nanoparticles deposited on a piece of Si substrate was collected by Siemens D-500 Diffractometer with Cu source (FIG. 8(d)). Diffraction peaks are consistent with the standard powder diffraction file of Fe₅Si₃, as well as Fe₅Si₃ compound fabricated by other methods. Above evidence supports the fact that Fe₅Si₃ phase is achieved using the physical gas condensation method.

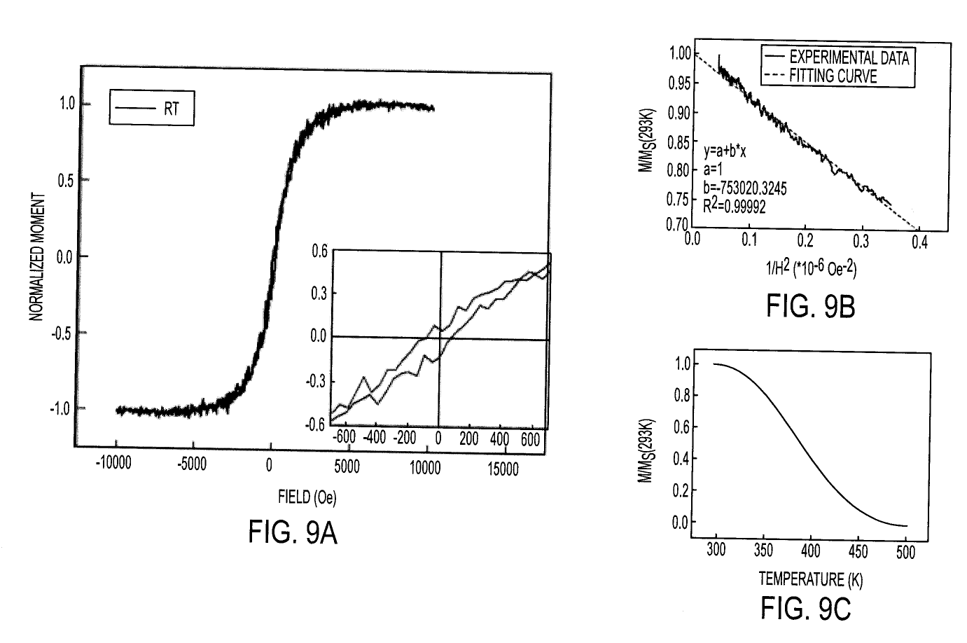

The magnetic property of the nanoparticles was characterized by vibrating sample magnetometer (VSM). A room temperature hysteresis loop was plotted in FIG. 9(a). It shows coercivity of about 90 Oe, which can be found from the inset zoom-in figure. Investigation on anisotropy constant was performed to understand the ferromagnetic hysteresis. In FIG. 9(b), initial magnetization curve under room temperature was fitted according to law of approach to saturation for uniaxial materials:

$$M = {M}_{s} ( 1 - \frac{b}{{H}^{2}} ) , \\ b = \frac{4 {K}^{2}}{15 {M}_{s}^{2}} .$$

By using Mₛ=358 emu/cm³, K was calculated to be 6.02×10⁵ erg/cm³. This is a reasonable value because hexagonal Fe₅Si₃ is a magnetically hard phase among soft silicides. Considering the size of the Fe₅Si₃ nanoparticles, they are not in the superparamagnetic range. Temperature dependence of magnetization was also measured by VSM under 1 T constant magnetic field during the entire measurement. FIG. 9(c) displays magnetization change as a function of temperature. Magnetization starts to drop quickly when temperature reaches approximate 350K. Extrapolation of M-T curve according to the power law M(T) ∝(1−T/T_{c})^{β}, β=0.36 gives Curie temperature 443K, which is higher compared to that of bulk. This is suspected to be caused by deviation from the exact stoichiometry for the ensemble of nanoparticles.

With heating by magnetic nanoparticles, the heating power is very sensitive to anisotropy constant. If anisotropy constant is away from the optimum value determined by the AC field parameter much, the heating effect from nanoparticles become negligible. Therefore, temperature dependence of anisotropy constant is even critical. For uniaxial metal alloys,

$$\frac{K ( T )}{K ( 0 )} = {[ \frac{M ( T )}{M ( 0 )} ]}^{n} ( n = 2 \sim 3 )$$

is valid. Hence much steeper drop of anisotropy constant could be expected for Fe₅Si₃ nanoparticles. Even before reaching the Curie point, as the K value deviates much from the ideal value with respect to the AC magnetic field parameters, power loss of nanoparticles would vanish. Consequently, over-heating could be eliminated effectively and damage to normal cells could thus be greatly reduced.

For an application that may need higher or lower regulation temperature, we can engineer the size and shape of FeSi particles and/or form the core-shell or heterostructure with Fe₅Si₃ as the core or embedded cluster in Fe or Fe₃Si matrix. The Curie temperature can be adjusted by engineering the ratio of the core-shell and other materials in the heteterostructured particle.

The regulation temperature (Curie temperature) can be controlled from room temperature up to 200° C. The matrix material or part-of core-shell material could be any efficient materials (e.g. Ag, Au, etc) for thermal ablation, which responses to the radio frequency electromagnetic field.

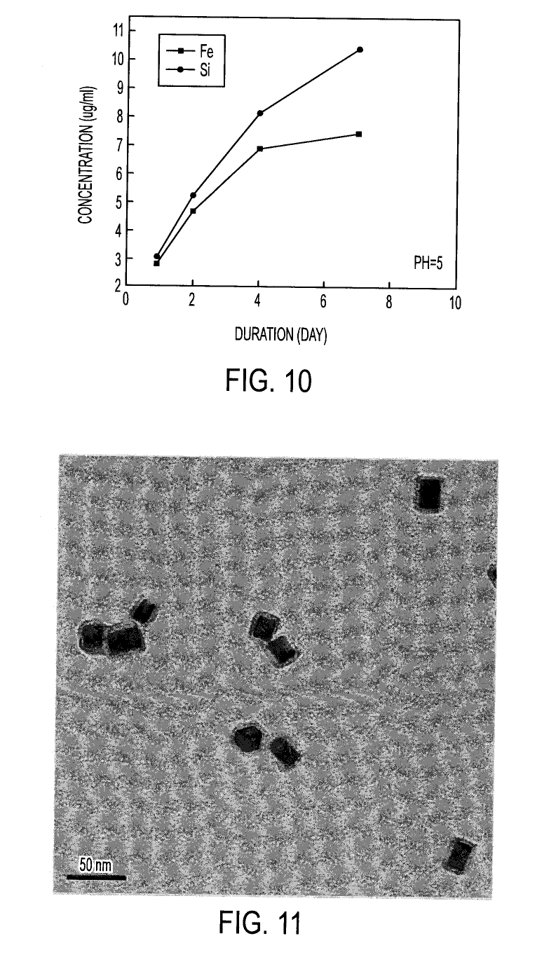

In order to apply the nanoparticles for in-vivo use, biodegradability is a feature that will assist the exertion of nanoparticles out of human body and leaving no long-term side-effects. Biodegradability of Fe₅Si₃ nanoparticles were tested in-vitro under simulated biological condition. PH=5 acetic acid and sodium acetic buffer was used to make the solution. This slightly acidic condition simulates lysosomal environment or tumor tissue environment. The solution was kept under 37 Celsius degree water bath. In periodic time interval, the solution was taken out and centrifuged. Supernatant was collected into a microtube for ICP analysis. FIG. 10 shows the ICP results of Fe and Si ions in the supernatant with respect to the time duration. The increasing ion concentration proved that Fe₅Si₃ nanoparticles can degrade under this in-vivo condition. And the two elements seem to follow different degradation rate. A complete degradation of nanoparticles is not reached at day 7. Further exploration of the degradation mechanism and total time length for complete degradation will be conducted. Nevertheless, the preliminary data gives evidence of biodegradable Fe₅Si₃ nanoparticles.

Biocompatible and biodegradable Fe₅Si₃ with relatively promising magnetization value is proposed for self-regulated magnetic hyperthermia. Fe₅Si₃ nanoparticles are made with narrow size distribution using a unique physical gas condensation method. These nanoparticles show ferromagnetic behavior at room temperature with anisotropy constant K=6.02×10⁵ erg/cm³. Fast decrease of magnetization under increased temperature and Curie temperature of 443K were found. When we take advantage of the high temperature sensitivity of anisotropy constant, desirable self-regulation performance could be expected from Fe₅Si₃ nanoparticles.

Nanotechnology has the potential to revolutionize medical diagnosis and treatment with magnetic nanoparticles emerging at the forefront of the field. These systems enable the localized delivery of thermal energy, which can induce cell death in malignant tumors sensitive to a moderate temperature increase or stimulate the release of drug from temperature-sensitive carriers. This approach overcomes the drawbacks of currently available therapies by exposing only the diseased region to activated particles. For such applications, magnetic NPs would ideally work in a designed, narrow temperature range and thereby would have minimal toxicity on normal cells.

A major difficulty in achieving the ideal thermal profile in vivo arises from the inability for thermal sensors to map the temperature profile accurately given the small spatial distribution. Nevertheless, it was shown by Huang et al. using fluorophore tags on the surface of magnetite nanoparticles that nanoparticles will induce more than a 15° C. temperature increase in a very short time at cell surface. While providing quantitative information, this approach does not appear adaptable for manual manipulation of the external electrical power to control the local temperature gradient. Thus, an alternative approach is desired that allows self-regulated heat generation.

One possibility is the use “smart” nanoparticles that exploit the Curie temperature (T_{c}) of magnetic material. The rate of heating slows as the Curie temperature is approached and no heating occurs at and above this temperature. This has been demonstrated with a number of ferrite materials, such as La₁₋ₓSrₓMnO₃, and Zn ferrite. However, these materials suffer from a low saturation magnetization. Since the heating rate is a function of the magnetic moment per particle, which is a product of its magnetization and volume, these ferrites do not produce sufficient heat for biomedical applications. Although it is possible to increase the magnetic moment by doping with rare earth elements, such additions result in particles with unacceptably toxicity. These include Ni—Cu, Fe—Ni, FeNiZrB, FeCoCr and Ni—Cr. A composite structure, such as a core-shell structure, could be employed to enhance the biocompatibility of, but significant concerns remain with respect to ensuring complete coverage of the toxic particles as well as the ultimate removal from the body. Thus, producing “smart” NPs composed of biologically acceptable components remains an unrealized goal.

To produce a functional material with intrinsic temperature regulation, Si is incorporated into Fe to produce the desired particles. It is recognized that the Curie temperature is determined by the strength of quantum mechanical exchange-coupling between Fe atoms. Moreover, investigations into bulk samples of Fe—Si alloys have revealed that hybridization between Fe and Si is possible, and this has influence on the observed magnetic property. As such, the underlying physics justify the use of Si to reduce T_{c} of Fe by tuning the interaction through a control of the exchange process. Moreover, Fe and Si are relatively nontoxic and therefore can be expected to be processed to produce a biocompatible material.

For the tuning of Fe—Si composites, it is critical that the mechanism of heat generation be understood as well as the quantitative influence of the magnetic properties of NPs and alternating magnetic field. The vanishing magnetic moment of low T_{c} materials has been addressed, but another important aspect is related to the magnetic anisotropy of NPs. This often overlooked property can be even more sensitive to temperature than that of magnetization. Specifically, when the magnetic anisotropy is outside the optimal heating performance range of the alternating magnetic field, negligible heat is generated.

In one or more embodiments, Fe—Si NPs of different compositions in pursuit of nanoparticles with adjustable temperature-sensitive magnetic properties were produced. A gas phase synthetic method was used to fabricate the Fe—Si nanoparticles, which allowed control on thermal environment for crystal growth, diffusion and segregation, and thereby facilitated fabrication of a wide range of NPs. In one or more embodiments, the method provides an adjustment of composition and exquisite control of the resulting crystalline phase, which is superior to traditional wet-chemical methods for preparation of nanoparticles.

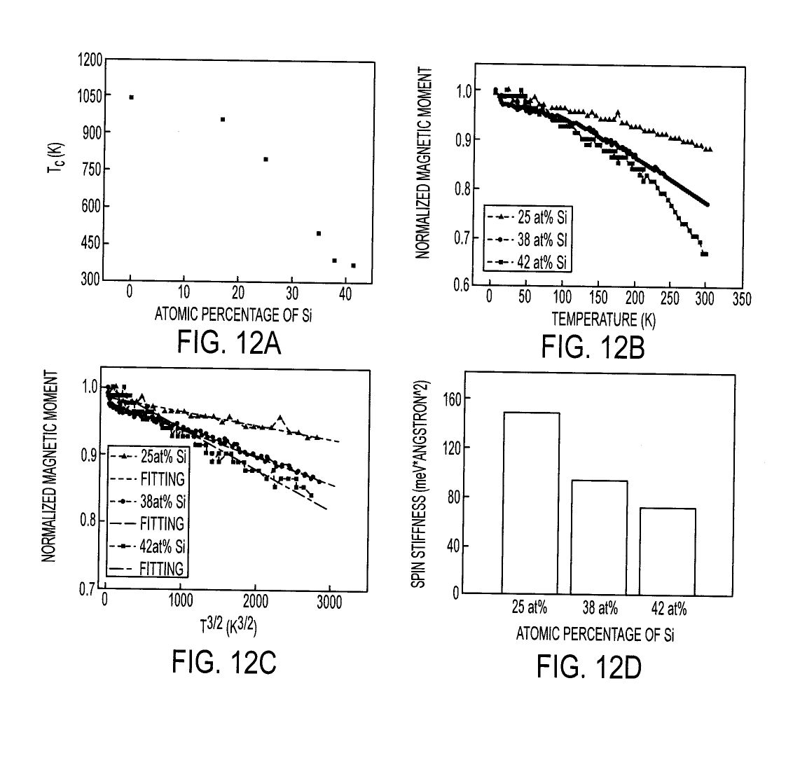

FIG. 11 displays transmission electron microscope (TEM) bright field image of Fe—Si nanoparticles that were prepared with 42 at % Si. The particles were mostly multi-faceted shaped, with good crystallinity and relatively high uniformity. The temperature dependence of saturation magnetization and associated Curie temperature were measured by a vibrating sample magnetometer (VSM). A constant magnetic field of 1 T was applied during measurement. T_{c} was determined from the tangential slope of the curve at the point of the ferromagnetic transition. It can be seen that T_{c} decreases as the atomic percentage of Si increased (FIG. 12a). A T_{c} in the range of 373-393 K was found for a Fe—Si NPs composed of 40±2 mol % Si. The results demonstrate effective reduction in T_{c} and supports the premise that T_{c} can be tuned by incorporating Si into Fe.

To investigate how Si modulates the T_{c}, the spin-wave stiffness coefficient was assessed from measurements of the magnetization as a function of temperature for different Si concentrations in the range of 5K to 300K. The magnetization was evaluated with the spin-wave theory in the frame of Heisenberg's model²⁸ by fitting the curves to T^{3/2} law in the range of 5K-150K. The results are shown in FIG. 12c where the intercept was close to unity (0.97-0.99), and there was high confidence in the fit (>90%). Spin-wave stiffness, D, was determined from the spin-wave dispersion relationship hω=Dq² and was calculated from the slope, A,

A=2.612(V/S)(k/4πD)^{3/2}

where V is the atomic volume of a magnetic atom and S is the spin. The calculated D is given in FIG. 12d. A general trend of declining D with increasing atomic percentage of Si is evident. This indicates that the weakening of exchange interactions among Fe atoms is realized by incorporation of Si, and thus T_{c} is correspondingly tuned. Fe—Si nanoparticles may be engineered for temperature regulation.

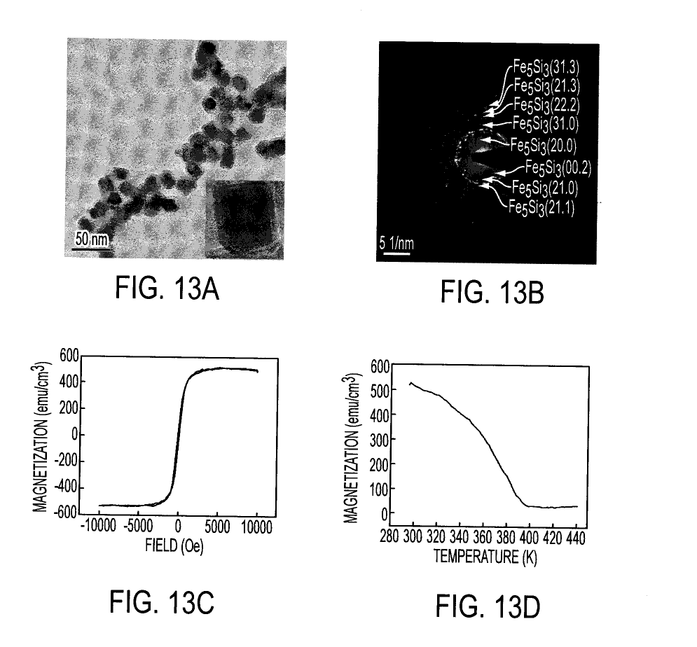

With the established capability of tuning T_{c} through a modulation of exchange interactions in Fe—Si, we then focused on nanoparticles of low T_{c}, which are amenable for self-regulated heat production with potential application in cancer hyperthermia or thermal stimulated drug release. FIG. 13a is a TEM bright field image of the cuboidal-shaped NPs with 38% Si. A high magnification image of a single particle is displayed in the inset, and FIG. 13b shows the respective selected area diffraction (SAD) pattern. The Fe—Si NPs are highly crystalline as evident from the discrete spots arising from the diffraction of the electron beam by the crystal planes. The SAD pattern also reveals that the Fe—Si NPs form Fe₅Si₃ with a hexagonal structure, consistent with the strong diffraction arising from (002) and (210) crystal planes. The hysteresis loop of an ensemble of nanoparticles was measured by VSM at room temperature (FIG. 13c). The saturation magnetization value was calculated based on the magnetic moment and the mass of particles and was 80 emu/g, or 536 emu/cm³ using a density of 6.7 g/cm³. It is higher than the previously proposed ferrite materials with low T_{c} and is comparable to the metallic alloy materials, such as magnetite. The remnant magnetic moment of the NPs at zero magnetic field was near 8.5% of saturation magnetic moment, while the coercivity was 70 Oe. Given the small particle size, no hysteresis was expected. It may be that the remnance and coercivity was a consequence of the interparticle interaction among NPs when packed together onto the substrate. In addition, some large particles in the sample may also contribute to the observed hysteresis. The temperature dependence of magnetization is depicted in FIG. 13d. A drop of magnetization takes place as the temperature increases, and a transition at about 393K is seen. To our knowledge, this is the first demonstration of a low T_{c} coupled with a relatively high saturation magnetization in a magnetic nanoparticle material that does not contain toxic, heavy metals.

The temperature dependence of the anisotropy constant was also investigated by measurement of the magnetization curves at different temperatures. The anisotropy constant was obtained by extrapolating the fitted magnetization curves to the law of approach to saturation magnetization for magnetic materials with uniaxial anisotropy. A decrease in the anisotropy constant was found as the temperature increased, and at room temperature, the effective anisotropy constant was 3×10⁵ erg/cm³. A square power law relationship was deduced by comparing the temperature dependence of anisotropy and magnetization.

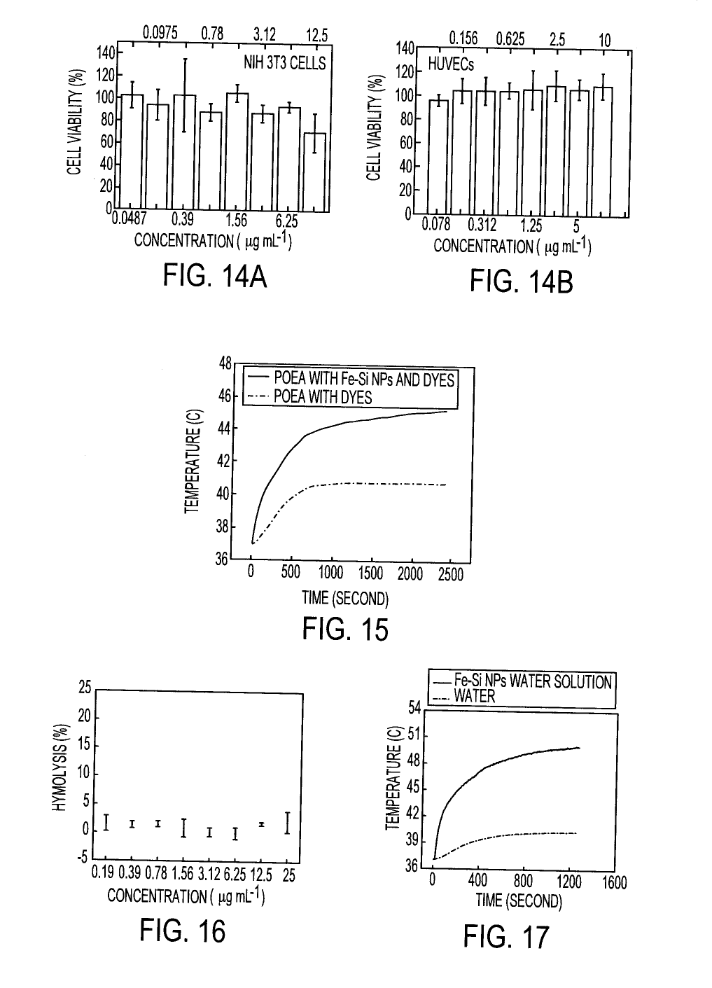

Following the characterization of the magnetic properties of the composite nanoparticles, the cytotoxicity and the rate of magnetic field heating was evaluated. To examine the biocompatibility of the Fe—Si NPs, the cytotoxicity was tested in cultured mouse embryonic fibroblasts (NIH 3T3) and human umbilical vein endothelial cells (HUVECs) using the standard MTT (3-(4,5-dimethylthiazol-2-yl)-2,5-diphenyltetrazolium bromide) assay. FIG. 14a shows that a high percentage of NIH 3T3 cells remained viable in concentrations of NP at 6.25 μg/mL. When the concentration of Fe—Si nanoparticles was increased to 12.5 μg/mL, the viability dropped to below 80%. Compared to other metallic nanoparticles, Fe—Si nanoparticles exhibited lower toxicity. Optical images were also taken of cells without and with the treatment of nanoparticles, and by comparison, the morphology of NIH 3T3 cells remained unchanged within the safe concentration range of nanoparticles. Similar results were observed for HUVECs (Supplementary FIG. S1). A hemolysis test was also carried out with different concentrations of Fe—Si NPs. The results show that particles failed to cause any damage to red blood cells as would be evident by leakage of hemoglobin (FIG. 16). Aggregation of blood cells was not observed under the testing conditions. These results indicate that Fe—Si NPs lack overt signs of toxicity, which is a promising indication for their use in medical applications.

Heat generation of Fe—Si NPs under an alternating magnetic field was assessed using a heat sink of 37° C. The specific loss power (SLP) was calculated from the initial temperature rise as a function of time. For the Fe—Si NPs suspended in phosphate buffered saline (PBS) pH=7.4, a SLP of about 209 W/g was obtained under an alternating magnetic field of 380 kHz and 40 Oe (See FIG. 17).

To test the possibility of using these particles for thermally stimulated drug delivery, a heating experiment was also performed in which the nanoparticles were incorporated into a thermosensitive block copolymer. A poly(ortho ester amides) (POEA) block copolymer was synthesized according to a published method so that a gel-sol transition temperature at about 45° C. was obtained. A composite sample was prepared by incorporating Fe—Si nanoparticles and a fluorecen, Rhodamine B dye into the POEA gel. A control sample of POEA gel loaded with only dye was also prepared as a control. In reviewing the sample with nanoparticles before the magnetic field was turned on, a clear difference of color can be seen between PBS buffer and the composite gel, which suggests that the dye is mostly entrapped within the gel. The temperature change of the composite during magnetic field heating was recorded. The composite gradually heats to the transition temperature of the block copolymer. Upon reaching the transition temperature, the gel dissolves into the PBS buffer and the dye was completely released into the solution. The final solution has a uniformly dark red color from the released dye. In comparison, the control sample without nanoparticles had an increase of temperature of about 3° C. (FIG. 15). Before and after magnetic field heating, a clear difference in color exists between the PBS buffer and the composite, which indicates that the gel did not dissolved in the absence of nanoparticles. The slight change in color of the PBS buffer before and after heating was likely due to passive diffusion of dye molecules from the surface of the composite. Overall, this experiment provides proof of the potential of Fe—Si nanoparticles for thermally stimulated drug delivery.

An innovative approach was used to prepare biocompatible “smart” magnetic nanoparticles for self-regulated therapeutic applications. Fe—Si nanoparticle system was used to demonstrate that a lower T_{c} can be achieved through tuning of exchange interaction. The lower T_{c} correlates with the weakening of exchange interaction as a consequence of the higher content of Si. Specifically, particles with composed of Fe₅Si₃ in a crystalline phase had a low T_{c} but retained a high magnetic moment. Thus, these nanoparticles represent a good candidate for self-regulated thermal therapy. The nanoparticles had relative low toxicity in cell cultures and demonstrated excellent heat generation that induced a phase transition in thermal sensitive block copolymers resulting in enhanced drug release. This work provides insights on a new type of biocompatible magnetic nanoparticles with self-regulation of temperature.

In one or more embodiments, a method for the synthesis of nanoparticles is provided herein. In one or more embodiments, Fe—Si magnetic nanoparticles are synthesized by a magnetron-sputtering-based nanocluster deposition system. Vapor of atoms was generated from the composite Fe—Si target by the sputtering gun. During the synthesis, sputtering current was in the range of 0.4 A-0.6 A. The pressure in the nanocluster source was varied between 300 mTorr and 600 mTorr by Ar gas feeding. The pressure in the deposition chamber was below 0.1 Pa. Nanoparticles were collected onto arbitrary substrates, such as a Si wafer, et al.

In a sample characterization, the morphology and structure of the nanoparticles were examined by TEM (FEI T12) and HRTEM (FEI G2 30). A VSM (Princeton measurements) equipped with a flowing helium gas furnace was used to probe magnetic properties at 300-800K. A magnetic property measurement system (Quantum Design, MPMS XL) was used to carry out the magnetic measurements at 5K-300K. The concentration of nanoparticle solution was determined by inductively coupled plasma spectrometry (ICP).

In one or more embodiments, an alternating magnetic field was generated by an induction coil system (Hyperthermia Inc.). The experimental samples were placed in the center of the coil with insulation wraps. The temperature change was monitored by a fluoroptic thermometry system (Luxtron 3100, Lumasense Technologies) and recorded by a computer.

In one or more embodiments, thermal sensitive gel is prepared with nanoparticle loading. A sample containing 0.015 g POEA block copolymer was heated above the transition temperature to form the sol. Concentrated Fe—Si nanoparticle s and 0.001 g Rhodamine B dyes in PBS were mixed with the sol. A control sample was made with only POEA and dyes in a similar manner. The sample was cooled down under shaking to form a gel. The concentration of nanoparticle was 0.5 wt % measured by ICP.

In a review of cytotoxicity, experiments were performed on NIH 3T3 cell line (ATCC) based on a standard procedure. Cell culture media was DMEM high glucose (Invitrogen) supplemented with 10% fetal bovine serum (FBS, heat inactivated, Gibco) and 100 units/mL penicillin/streptomycin (Gibco). Fe—Si nanoparticles with polyethylene glycol (PEG) surface coating were mixed with PBS buffer (Invitrogen) to reach a concentration of 0.125 mg mL⁻¹. Afterwards, this fresh stock solution of nanoparticles was diluted using PBS buffer to 0.0487 mg mL⁻¹. 20 μL of Fe—Si nanoparticle solution were added to each well of a 96-well plate containing NIH 3T3 cells seeded at 5000 cells/well in 180 μL cell culture media and was incubated at 37° C. in 5% CO₂ for 24 h before performing the MTT assay (Sigma) to determine cell viability in triplicates. To perform the assay, each well was washed gently using PBS twice, then 20 μL of MTT stock solution (5 mg mL⁻¹ in PBS) was added to the cells in each well and incubated for 4 h. Cell culture media was then removed and replaced with 150 μL of DMSO, and absorbance was read at 570 nm using a Synergy HT microplate reader (Bio-TEK). Cell viability was calculated by [Absorbance of cells exposed to nanoparticles]/[Absorbance of cells cultured without nanoparticles] in percentage. Cell images were captured before MTT was added using an Olympus IX70 upright microscope under polarized light. Similarly, cytotoxicity of HUVECs (ATCC) was also measured.

In a review of hemolysis, an eight-week old male mouse was sacrificed by CO₂ asphyxiation and blood was collected from the heart and centrifuged at 1500 rpm for 5 min at 4° C. The plasma was removed, and the erythrocytes were resuspended in 2 mL ice cold PBS. The cells were again centrifuged at 1500 rpm for 5 min at 4° C. This procedure was repeated more than twice to ensure the removal of any released hemoglobin. After the supernatant was removed, the cells were resuspended in PBS solution and diluted to obtain a cell suspension with Abs=0.6 at 650 nm. nanoparticles were also diluted in PBS to reach different concentrations. 0.1 mL of the nanoparticles were added to 0.1 mL of the RBC suspension in a 96-well plate and incubated for 1 h at 37° C. with mild shaking. Complete hemolysis was attained using a 2% v/v Triton-X yielding the 100% control value. PBS was used as negative control. After incubation, the 96-well plates were centrifuged at 1500 rpm for 5 min, and 100 μL of the supernatants were transferred to another 96 well plate. The release of hemoglobin was determined by UV at 414 nm. Each sample was measured in triplicates. Degree of hemolysis is defined as % lysis=100*(A_{NANOPARTICLE}−A_{blank})/(Aₜᵣᵢₜₒₙ−A_{blank})

### Magnetic Field Heating

Specific loss power of particles was calculated based on the rise of temperature and the equation below taking into consideration of background.

$$P = \frac{{m}_{fluid}}{{m}_{particle}} \frac{d T}{d t} c$$

where m represents either the mass of the fluid or the mass of nanoparticles,

$$\frac{d T}{d t}$$

is the initial rate of temperature rise, and c is the heat capacity of water (4.187 J g⁻¹K⁻¹).

Various methods related to the compositions of nanoparticles are as follows. In one or more embodiments, a method includes functionalizing the biocompatible and biodegradable nanoparticles with specific targeting groups for cells or tissues, the nanoparticles including crystalline alloy or partial-crystalline alloy or amorphous alloy structure, the nanoparticle includes an alloy of Fe and at least one of Mg, Zn, Si, N, C, or P; and delivering the nanoparticles, for instance, into human bodies, and imaging the nanoparticles using for example, but not limited to, contrast agencies for magnetic resonant imaging or other in-vivo imaging processes.

In one or more embodiments, a method includes functionalizing the biocompatible and biodegradable nanoparticles with specific molecules or their combinations for in-vivo medical devices, where the nanoparticles include crystalline alloy or partial-crystalline alloy or amorphous alloy structure, and the nanoparticles include an alloy of Fe and at least one of Mg, Zn, Si, N, C, or P. The method further includes controlling the nanoparticles by a magnetically heating process to release those molecules regularly and degrade the particles where the magnetic field generator could be external or internal the human bodies.

In one or more embodiments, a method includes functionalizing the biocompatible and biodegradable nanoparticles with specific molecules or their combinations, the nanoparticles including crystalline alloy or partial-crystalline alloy or amorphous alloy structure, where the nanoparticles include an alloy of Fe and at least one of Mg, Zn, Si, N, C, or P, and separating and sorting molecules or cells, and degrading the nanoparticles after usage thereof.

Further options include functionalizing the biocompatible and biodegradable nanoparticles with at least one of specific targeting groups for cells or tissues, or specific molecules or combinations for in-vivo medical devices. In a further option, the method further includes at least one of imaging with the nanoparticles, controlling the nanoparticles with a magnetic heating process to release molecules and to degrade the particles, sorting the nanoparticles.

In one or more embodiments, a method includes disposing iron on at least one of a Fe—Zn, Fe—Mg, Fe—Si, Fe—C, FeN or FeP composite target, disposing the Fe—Zn, Fe—Mg or Fe—Si, Fe—C, FeN or FeP target in a high vacuum chamber, generating related atoms or ions from the target in a sputtering process or evaporating process, and freezing nano-clusters and/or nanoparticles, for example, at an equilibrium or non-equilibrium phase in the sputtering process or evaporating process. In a further option, the method includes collecting nanoparticles and or their aggregates on organic or inorganic substrate or liquid.

In one or more embodiments, a method includes magnetically heating adjacent material with magnetic, biocompatible and biodegradable nanoparticles, the nanoparticles including crystalline alloy or partial-crystalline alloy or amorphous alloy structure, the nanoparticles include an alloy of Fe and at least one of Mg, Zn, Si, N, C, or P. The method further includes maintaining a predetermined temperature based on a magnetic property of the nanoparticles. In one or more embodiments, magnetization of the nanoparticles substantially decreases when a temperature of the nanoparticles reaches a predetermined value. In one or more embodiments, the nanoparticles cease heating up when a temperature of the nanoparticles reaches a predetermined value. In at least one embodiment, heating the nanoparticles includes heating the nanoparticles to a temperature 60 C-100 C suitable for ablations, or heating the nanoparticles to a temperature between 40° C. to 60° C. suitable for magnetic hyperthermia. The heating power, in an option, is sensitive to an exchange coupling constant between magnetic atoms in the nanoparticles, or a magnetocrystalline anisotropy constant of the nanoparticle. The method further optionally includes delivering drugs with the nanoparticles, or releasing drugs from the nanoparticles.

The following are incorporated herein by reference:
Rudolf Hergt, Silvio Dutz, Robert Müller and Matthias Zeisberger, J. Phys.: Condens. Matter 18, S2919-S2934 (2006)
Q A Pankhurst, J Connolly, S K Jones and J Dobson, J. Phys. D: Appl. Phys. 36, R167-R181 (2003)
Pawel Keblinski, David G. Cahill, Arun Bodapati, Charles R. Sullivan and T. Andrew Taton, J. Appl. Phys., 100, 054305 (2006)
Klaus Maier-Hauff, Ronny Rothe, Regina Scholz, Uwe Gneveckow, Peter Wust, Burghard Thiesen, Annelie Feussner, Andreas von Deimling, Norbert Waldoefner, Roland Felix and Andreas Jordan, J Neurooncol 81, 53-60 (2007)
Ivan A. Brezovich, William J. Atkinson and Dev P. Chakraborty, Med. Phys., 11(2), 145 (1984)
E. Pollert, K. KnÍ\check{z}ek, M. Mary\check{s}ko, P. Ka\check{s}par, S. Vasseur and E. Duguet, J. Magn. Magn. Mater., 316, 122-125 (2007)
K. L. McNerny, Y. Kim, D. E. Laughlin, and M. E. McHenry, J. App. Phys., 107, 09A312 (2010)
Tatiana N. Brusentsova, Nikolay A. Brusentsov, Viatcheslav D. Kuznetsov, Vladimir N. Nikiforov, J. Magn. and Magn. Mater., 293, 298-302 (2005)
Kumar S. K. Varadwaj, Kwanyong Seo, Juneho In, Paritosh Mohanty, Jeunghee Park, and Bongsoo Kim, J. Am. Chem. Soc., 129 (27), 8594-8599 (2007)
Di Chen, Gary N. Otto and Francis M. Schmit, IEEE Trans. Magn, MAG-9, 66 (1973)
Jiao-Ming Qiu and Jian-Ping Wang, Adv. Mater., 19, 1703-1706 (2007)
Jiao-Ming Qiu and Jian-Ping Wang, Appl. Phys. Lett., 88, 192505 (2006)
Li Tie, Li Yu-zhi and Zhang Yu-heng, J. Phys.: Condens. Matter, 9, 1381-1388 (1997)
R. William McCallum, J. Magn. Magn. Mater., 292 135-142 (2005)
G. Herzer, IEEE Trans. Magn., 25, 3327 (1989)
A Belayachi, J L Dormann and M Nogues, J. Phys.: Condens. Matter 10, 1599-1619 (1998)
H. Amekura, Y. Fudamoto, Y. Takeda, and N. Kishimoto, Phys. Rev. B, 71, 172404 (2005)
Hweerin Sohn and R. H. Victora, J. Appl. Phys. 107, 09B312 (2010)
J. B. Staunton, L. Szunyogh, A. Buruzs, B. L. Gyorffy, S. Ostanin and L. Udvardi, Phys. Rev. B 74, 144411 (2006)

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope of the invention should, therefore, be determined with reference to the appended claims, along with the full scope of equivalents to which such claims are entitled.

What is claimed is:

1. A method for inducing cell death in a tumor, the method comprising:
magnetically heating biocompatible and biodegradable nanoparticles that are adjacent to the tumor to induce cell death in the tumor.

2. The method of claim 1, wherein the nanoparticle comprises an alloy of Fe and at least one of Mg, or Zn.

3. The method of claim 1, wherein the nanoparticle is maintained at a predetermined temperature to induce cell death in the tumor.

4. The method of claim 3, maintaining the predetermined temperature is based on a magnetic property of the nanoparticles.

5. The method of claim 1, wherein the nanoparticles cease heating when a temperature of the nanoparticles reaches a predetermined value.

6. The method of claim 1, wherein heating the nanoparticles includes heating the nanoparticles to a temperature between 60° C.-100° C.

7. The method of claim 1, wherein heating the nanoparticles includes heating the nanoparticles to a temperature between 40° C. to 60° C.

8. The method of claim 1, further comprising delivering a drug with the nanoparticles.

9. The method of claim 1, further comprising releasing a drug from the nanoparticles.

10. The method of claim 1, wherein magnetization of the nanoparticles substantially decreases when a temperature of the nanoparticles reaches a predetermined value.

11. The method of claim 1, wherein heating power of the nanoparticles is sensitive to an exchange coupling constant between magnetic atoms in the nanoparticles.

12. The method of claim 1, wherein heating power of the nanoparticles is sensitive to a magnetocrystalline anisotropy constant of the nanoparticles.

13. The method of claim 1, further comprising imaging the nanoparticles with magnetic resonant imaging.
